SEQUENCE LISTING (15 sequences)

SEQ ID NO: 1 - DNA, 3603 bp, synthetic construct
  misc_feature 1..3603: note = Description of artificial sequence: Del 1-3 ATP7B, native
  misc_feature 223..225: note = Nucleotides 223-225 encode for serine residue S340 (numbering based on wild-type protein sequence)
  misc_feature 226..228: note = Nucleotides 226-228 encode for serine residue S341 (numbering based on wild-type protein sequence)
  source 1..3603: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcagggatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 2 - DNA, 145 bp, synthetic construct
  misc_feature 1..145: note = Description of artificial sequence: AAV2 ITR
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 3 - DNA, 100 bp, synthetic construct
  misc_feature 1..100: note = Description of artificial sequence: enTTR Enhancer sequence
  source 1..100: mol_type = other DNA
ctacctcgtgatcgcccggcccctgttcaaacatgtcctaatactctgtctctgcaagggtcatcagtagttttccatcttactcaacatcctcccagtg

SEQ ID NO: 4 - DNA, 95 bp, synthetic construct
  misc_feature 1..95: note = Description of artificial sequence: SV40 Small T Intron
  source 1..95: mol_type = other DNA
gctctaaggtaaatataaaatttttaagtgtataatgtgttaaactactgattctaattgtttctctcttttagattccaacctttggaactgat

SEQ ID NO: 5 - DNA, 315 bp, synthetic construct
  misc_feature 1..315: note = Description of artificial sequence: rHBB Intron
  source 1..315: mol_type = other DNA
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctcaagagcctctgctaaccatgttcatgccttcttctttttcctacagctcctgggcaacgtgctggttattgtgctgtctcatcattttggcaaagaatt

SEQ ID NO: 6 - DNA, 215 bp, synthetic construct
  misc_feature 1..215: note = Description of artificial sequence: BGH polyadenylation signal sequence
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 7 - DNA, 198 bp, synthetic construct
  misc_feature 1..198: note = Description of artificial sequence: SV40 polyadenylation signal sequence
  source 1..198: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttag

SEQ ID NO: 8 - protein, 1200 aa, synthetic construct
  REGION 1..1200: note = Description of artificial sequence: DEL 1-3 ATP7B Native, protein
  SITE 75: note = MISC_FEATURE - Amino acid residue at position 75 corresponds to Serine S340 (numbering based on wild-type protein sequence)
  SITE 76: note = MISC_FEATURE - Amino acid residue at position 76 corresponds to Serine S341 (numbering based on wild-type protein sequence)
MPEQERQITAREGASRKILSKLSLPTRAWEPAMKKSFAFDNVGYEGGLDGLGPSSQVATSTVRDGAEGSGTDHRSSSSHSPGSPPRNQVQGTCSTTLIAIAGMTCASCVHSIEGMISQLEGVQQISVSLAEGTATVLYNPSVISPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 9 - DNA, 2211 bp, synthetic construct
  misc_feature 1..2211: note = Description of artificial sequence: Nucleic acid sequence encoding AAV9 capsid protein VP1
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 10 - protein, 736 aa, synthetic construct
  REGION 1..736: note = Description of artificial sequence: Amino acid sequence encoding AAV9 capsid protein VP1
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - skipped

SEQ ID NO: 12 - DNA, 190 bp, synthetic construct
  misc_feature 1..190: note = Description of artificial sequence: TTR promoter Sequence
  source 1..190: mol_type = other DNA
atttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 13 - protein, 1120 aa, synthetic construct
  REGION 1..1120: note = Description of artificial sequence: DEL 1-4 ATP7B Native, protein
MPEQERQITAREGASRKILSKLSLPTRAWEPAMKKSFAFDNVGYEGGLDGLGPSSQVATSTVRSPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 14 - DNA, 4485 bp, synthetic construct
  misc_feature 1..4485: note = Description of artificial sequence: Expression cassette of exemplary vector DTC319
  source 1..4485: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctacctcgtgatcgcccggcccctgttcaaacatgtcctaatactctgtctctgcaagggtcatcagtagttttccatcttactcaacatcctcccagtgatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctctaaggtaaatataaaatttttaagtgtataatgtgttaaactactgattctaattgtttctctcttttagattccaacctttggaactgatgccgccaccatgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcagggatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 15 - DNA, 3603 bp, synthetic construct
  misc_feature 1..3603: note = Description of artificial sequence: Del 1-3 ATP7B, codon-optimized
  source 1..3603: mol_type = other DNA
atgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctga

(19) United States
(12) Patent Application Publication
(10) Pub. No.: US 2025/0333763 A1
(43) Pub. Date: Oct. 30, 2025

(54) GENE THERAPY CONSTRUCTS FOR TREATING WILSON DISEASE

(71) Applicant: Ultragenyx Pharmaceutical Inc., Novato, CA (US)

(72) Inventors: Christine Livingston, Lambertville, NJ (US); Samuel Wadsworth, Monterey, MA (US)

(21) Appl. No.: 19/213,623

(22) Filed: May 20, 2025

Related U.S. Application Data
(63) Continuation of application No. 17/417,619, filed on Jun. 23, 2021, now Pat. No. 12,338,450.
(60) Provisional application No. 62/788,324, filed on Jan. 4, 2019.
(60) Provisional application No. 62/834,830, filed on Apr. 16, 2019.

(51) Int. Cl. C12N 15/86; A61K 48/00; A61P 43/00

(52) U.S. Cl. CPC C12N 15/86; A61K 48/0058; A61P 43/00

(57) ABSTRACT

This application relates to adeno-associated viral vectors encoding a truncated yet functional ATP7B for use in gene therapy for treating Wilson disease (WD). The truncated ATP7B described herein has several advantages over the wild-type ATP7B such as higher efficacy and improved manufacturing yield.

### CROSS-REFERENCE TO RELATED APPLICATIONS

This application is a continuation application of U.S. patent application Ser. No. 17/417,619, filed on Jun. 23, 2021, which application is a U.S. national stage application, filed under 35 U.S.C. § 371, of International Application No. PCT/US2020/012131, filed on Jan. 3, 2020, which claims the benefit of and priority to U.S. Provisional Application No. 62/788,324, filed on Jan. 4, 2019; and to U.S. Provisional Application No. 62/834,830, filed on Apr. 16, 2019, the entire disclosure of each of which are incorporated herein by reference for all purposes.

### SEQUENCE LISTING

The instant application contains a Sequence Listing which has been submitted electronically in ASCII format and is hereby incorporated by reference in its entirety. Said ASCII copy, created on May 14, 2025, is named ULTA-013USC1_SL.xml and is 39,435 bytes in size.

### TECHNICAL FIELD OF THE INVENTION

This application relates generally to adeno-associated viral vectors and methods of their use in gene therapy for treating Wilson disease (WD).

### BACKGROUND OF THE INVENTION

Wilson disease (WD) is an autosomal recessive genetic disorder that causes accumulation of copper primarily in the liver and subsequently in the neurological system and other tissues. WD is a rare disorder that affects approximately 1 in 30,000 individuals, caused by mutations in the copper transporting ATPase 2 (ATP7B) gene on chromosome 13. There are more than 600 unique ATP7B mutations. ATP7B is expressed mainly in hepatocytes and functions in the transmembrane transport of copper. Absent or reduced function of ATP7B protein results in decreased hepatocellular excretion of copper into bile, causing liver disease. Over time without proper treatment, high copper levels can cause life-threatening organ damage.

Patients with hepatic WD usually present in late childhood or adolescence, and exhibit features of acute hepatitis, fulminant hepatic failure, or progressive chronic liver disease. Neurologic manifestations of WD typically present later than the liver disease, most often in the second or third decade and include extrapyramidal, cerebellar, and cerebral-related symptoms.

The aim of medical treatment of WD is to remove the toxic deposit of copper from the body and to prevent its reaccumulation. Current treatment approaches for WD are daily oral therapy with chelating agents (D-penicillamine, trientine, and zinc salts). Medical therapy is effective in most, but not all WD patients. Liver transplantation is a therapeutic option in WD patients presenting with fulminant liver failure or progressive liver failure. However, transplant recipients are required to maintain a constant immune suppression regimen to prevent rejection.

The present invention addresses the need for improved and sustainable treatment of WD by delivering a gene expressing truncated yet functional ATP7B to patients with an adeno-associated viral vector. The truncated ATP7B of the present invention has improved efficacy in treating WD and possesses an advantage of manufacturing ease and efficiency over wild-type and other truncated forms of ATP7B protein.

### SUMMARY OF THE INVENTION

This invention provides compositions and methods of their use in gene therapy. Provided herein are adeno-associated virus (AAV) vectors useful for the treatment of WD. In one aspect, the present invention provides a recombinant nucleic acid construct comprising: a 5′-inverted terminal repeat (ITR) sequence; a promoter sequence; a nucleic acid sequence encoding a truncated human copper-transporting ATPase 2 (ATP7B) in which metal-binding domains (MBDs) 1-3 have been deleted, but the serine-rich loop including two serine residues (S340 and S341) between MBD3 and MBD4 is present; and a 3′-ITR sequence.

In another aspect, the present invention provides a recombinant adeno-associated virus (rAAV) useful for the treatment of Wilson disease, in which rAAV comprises an AAV capsid and a vector genome packaged therein, the vector genome comprises a 5′-inverted terminal repeat (ITR) sequence; a promoter sequence; a nucleic acid sequence encoding a truncated human copper-transporting ATPase 2 (ATP7B) in which metal-binding domains (MBDs) 1-3 have been deleted, but the serine-rich loop including two serine residues (S340 and S341) between MBD3 and MBD4 is present; and a 3′-ITR sequence.

These and other aspects and features of the invention are described in the following sections of the application.

### BRIEF DESCRIPTION OF THE DRAWINGS

The invention can be more completely understood with reference to the following drawings.

FIG. 1 is an illustrative diagram showing an exemplary vector genome construct comprising a nucleotide sequence, which encodes a truncated human copper-transporting ATPase 2 (ATP7B), in which the metal-binding domains (MBDs) 1-3 have been deleted, but the serine-rich loop including two serine residues (S340 and S341) between MBD3 and MBD4 is present (“ATP7B Δ1-3-SS” or “ATP7B del1-3 native”). Features of the exemplary vector genome construct are provided below:

| Start (Nucleotide | End (Nucleotide | |
|---|---|---|
| Position) | Position) | Description |
| 1 | 145 | Inverted terminal repeat |
| | | (ITR) |
| 146 | 245 | Enhancer |
| 246 | 435 | Promoter |
| 436 | 530 | Intron |
| 531 | 536 | Consensus Kozak sequence |
| 540 | 4142 | ATP7B dell-3 native |
| | | (wild-type) cDNA |
| 4143 | 4340 | Poly A signal |
| 4341 | 4485 | Inverted terminal repeat |
| | | (ITR) |

FIG. 2 is a schematic representation of an exemplary AAV vector (DTC319), with various key components shown therein.

FIG. 3 is a schematic representation of an exemplary plasmid, pAAV2/8.KanR (p2123FH) AAV Rep/Cap plasmid, which provides Rep and Cap function in packaging rAAV when co-transfected with AAV vectors into host cells.

FIG. 4 is a schematic representation of an exemplary plasmid, pAdDeltaF6 (Kan) adenovirus helper plasmid, for rAAV production when co-transfected with AAV vectors and Rep/Cap plasmids into host cells.

FIG. 5 is a scatter plot of liver copper (μg/g) in C3He-Atp7b^{tx-j} female mice (represented by circles) injected with either 10⁹, 10¹⁰, or 10¹¹ genome copies (GC)/kg of ATP7BcoFL (full-length human ATP7B which has been codon-optimized) and C3He-Atp7b^{tx-j} male mice (represented by squares) injected with either 10¹⁰ or 10¹¹ GC/kg of the same vector. Copper levels from age-matched uninjected male and female heterozygous (Het) and C3He-Atp7b^{tx-j} mice are also represented in the scatter plot.

FIG. 6 is a bar graph showing total yield rAAV (titers in GC) produced from host cells after transfection of AAV vectors encoding full or partial coding sequences of human ATP7B (AAV vector carrying nucleotide sequence for encoding full-length (FL) human ATP7B; AAV vector carrying nucleotide sequence for encoding human ATP7B in which MBDs 1-3 have been deleted, but the serine-rich loop including two serine residues (S340 and S341) between MBD3 and MBD4 is present (ATP7B Δ1-3-SS); or AAV vector carrying nucleotide sequence for encoding human ATP7B in which MBDs 1-4 have been deleted (ATP7B Δ1-4).

FIG. 7 is a scatter plot of urine and liver copper levels, squares and circles, respectively (μg/g), assayed after C3He-Atp7b^{tx-j} mice were injected with AAV8 carrying full-length human ATP7B (ATP7B FL), ATP7B Δ1-3-SS, or ATP7B Δ1-4. Phosphate-buffered saline (PBS) administered C3He-Atp7b^{tx-j} mice served as controls (vehicle).

FIG. 8 is a bar graph showing total yield rAAV (titers in GC) produced from host cells after transfection of AAV vector (DTC319) that encodes a truncated human ATP7B, in which metal-binding domains (MBDs) 1-3 have been deleted, but the serine-rich loop including two serine residues (S340 and S341) between MBD3 and MBD4 is present, encoding either AAV8 or AAV9 capsid.

FIG. 9 is a bar graph of liver copper accumulation levels (μg/g dry weight) in C3He-Atp7b^{tx-j} mice that were administered an intravenous injection of a vehicle control (dilution buffer; WD) or an infusion of AAV8 carrying native ATP7B Δ1-3-SS (DelA). Liver copper accumulation levels in uninjected wild-type mice (WT) represented in the bar graph served as a negative control. Values expressed as mean±SEM (standard error of the mean).

FIG. 10 is a bar graph of ceruloplasmin activity in C3He-Atp7b^{tx-j} mice that were administered an intravenous injection of a vehicle control (dilution buffer; WD) or an infusion of AAV8 carrying native ATP7B Δ1-3-SS (DelA), as measured by an enzymatic reaction-based colorimetric activity assay. Ceruloplasmin activity in uninjected wild-type (WT) mice, as measured by the same enzymatic reaction-based colorimetric activity assay is also represented in the bar graph.

FIG. 11 is a bar graph of the average score after standard assessment of Hematoxylin and Eosin (H&E) slides for nuclear enlargement and hepatocellular hypertrophy, disorganization, inflammatory infiltrate, and hepatocellular necrosis.

FIG. 12 is an illustrative diagram showing an exemplary vector genome construct DTC327 comprising an AAV9 capsid with PPIA polyA, AAV2 Rep/ITRs with full p5 promoter comprising 145 bps ITRs, and a nucleotide sequence encoding a truncated human copper-transporting ATPase 2 (ATP7B), in which the metal-binding domains (MBDs) 1-3 have been deleted, but the serine-rich loop including two serine residues (5340 and 5341) between MBD3 and MBD4 is present.

### DETAILED DESCRIPTION OF THE INVENTION

This invention provides agents and compositions for use in treating Wilson disease (WD). The nucleic acid sequences, vectors, recombinant viruses, and associated compositions of this invention as described herein can be used for ameliorating, preventing, or treating WD.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 12 recognized serotypes of AAV (AAV1-12).

Administration/Administer: To provide or give a subject an agent, such as a therapeutic agent (e.g., a recombinant AAV), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal, and inhalation routes.

ATP7B Δ1-3-SS: As used herein, ATP7B Δ1-3-SS refers to a truncated human copper-transporting ATPase 2 (ATP7B), in which the metal-binding domains (MBDs) 1-3 have been deleted, but the serine-rich loop including two serine residues (S340 and S341) between MBD3 and MBD4 is present.

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Enhancer: A nucleic acid sequence that increases the rate of transcription by increasing the activity of a promoter.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are required for vector encapsidation.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease (such as WD) refers to inhibiting the full development of a disease. “Treating” refers to a therapeutic intervention that ameliorates a sign or symptom of a disease or pathological condition (such as WD) after it has begun to develop. “Ameliorating” refers to the reduction in the number or severity of signs or symptoms of a disease (such as WD).

Promoter: A region of DNA that directs/initiates transcription of a nucleic acid (e.g., a gene). A promoter includes necessary nucleic acid sequences near the start site of transcription.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” refers to an AAV particle in which a recombinant nucleic acid molecule such as a recombinant nucleic acid molecule encoding a truncated human ATP7B (e.g., SEQ ID NO:1 or SEQ ID NO:15) has been packaged.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970: Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Rio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Serotype: A group of closely related microorganisms (such as viruses) distinguished by a characteristic set of antigens.

Stuffer sequence: Refers to a sequence of nucleotides contained within a larger nucleic acid molecule (such as a vector) that is typically used to create desired spacing between two nucleic acid features (such as between a promoter and a coding sequence), or to extend a nucleic acid molecule so that it is of a desired length. Stuffer sequences do not contain protein coding information and can be of unknown/synthetic origin and/or unrelated to other nucleic acid sequences within a larger nucleic acid molecule.

Subject: Living multi-cellular vertebrate organisms, a category that includes human and non-human mammals.

Synthetic: Produced by artificial means in a laboratory, for example a synthetic nucleic acid can be chemically synthesized in a laboratory.

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Viral Vectors:

In some aspects, the present disclosure provides a recombinant adeno-associated virus (AAV) vector containing a genome comprising an AAV 5′-inverted terminal repeat (ITR) sequence, a promoter sequence, a nucleic acid sequence which encodes ATP7B Δ1-3-SS (e.g., SEQ ID NO:1 or SEQ ID NO:15), and an AAV 3′-inverted terminal repeat sequence (ITR).

In some embodiments, the genome may further comprise an enhancer, an intron, a consensus Kozak sequence, and/or a polyadenylation signal as described herein. In some embodiments, the recombinant vector can further include one or more stuffer nucleic acid sequences. In one embodiment, a stuffer nucleic acid sequence is situated between the intron and the partial or complete coding sequence for ATP7B.

In various embodiments described herein, the recombinant virus vector is an adeno-associated virus (AAV) vector. The AAV vector can be an AAV vector of serotype 1, 2, 3, 4, 5, 6, 7, 8, 9, 10, 11, or 12 (i.e., AAV1, AAV2, AAV3, AAV4, AAV5, AAV6, AAV7, AAV8, AAV9, AAV10, AAV11, or AAV12), as well as any one of the more than 100 variants isolated from human and nonhuman primate tissues. See, e.g., Choi et al., 2005, Curr Gene Ther. 5: 299-310, 2005 and Gao et al., 2005, Curr Gene Ther. 5: 285-297. AAV vectors of any serotype may be used in the present invention, and the selection of AAV serotype will depend in part on the cell type(s) that are targeted for gene therapy. For treatment of WD, the liver is one of the relevant target organs. In some embodiments, the AAV vector is selected from serotype 9 (AAV9), serotype 8 (AAV8), serotype 5 (AAV5), or variant thereof. In an exemplary embodiment, the AAV vector is serotype 9 (AAV9) or a variant thereof.

In some embodiments, the recombinant AAV vector includes an AAV ITR sequence, which functions as both the origin of vector DNA replication and the packaging signal of the vector genome, when AAV and adenovirus helper functions are provided in trans. Additionally, the ITRs serve as the target for single-stranded endonucleatic nicking by the large Rep proteins, resolving individual genomes from replication intermediates.

In some embodiments, the 5′-ITR sequence is from AAV2. In some embodiments, the 3′-ITR sequence is from AAV2. In some embodiments, the 5′-ITR sequence and the 3′-ITR sequence are from AAV2. In some embodiments, the 5′-ITR sequence and/or the 3′-ITR sequence are from AAV2 and comprise or consist of SEQ ID NO:2. In other embodiments, the 5′-ITR sequence and/or the 3′-ITR sequence are from a non-AAV2 source.

In some exemplary embodiments, the AAV vector is an AAV serotype 9 (AAV9) vector, and the vector includes an enhancer, a promoter, an intron, a nucleic acid sequence which encodes ATP7B Δ1-3-SS (e.g., SEQ ID NO:1 or SEQ ID NO:15), and a polyadenylation signal described herein. In some embodiments, the AAV9 vector further includes two AAV2, AAV8, or AAV9 inverted terminal repeat (ITR) sequences: one 5′ of the enhancer and another 3′ of the polyadenylation signal. In an exemplary embodiment, the AAV9 vector includes two AAV2 inverted terminal repeat (ITR) sequences: one 5′ of the enhancer and another 3′ of the polyadenylation signal. In some embodiments, the AAV2 ITR sequences comprise or consist of SEQ ID NO:2. In another exemplary embodiment, the AAV9 vector includes two AAV9 inverted terminal repeat (ITR) sequences: one 5′ of the enhancer and another 3′ of the polyadenylation signal.

In some exemplary embodiments, the present disclosure provides a recombinant nucleic acid comprising a vector genome comprising an AAV 5′-inverted terminal repeat (ITR) sequence, a promoter sequence, a nucleic acid sequence represented by SEQ ID NO:1, which encodes native ATP7B Δ1-3-SS, and an AAV 3′-inverted terminal repeat sequence (ITR). In some exemplary embodiments, the present disclosure provides a recombinant nucleic acid comprising a vector genome comprising an AAV 5′-inverted terminal repeat (ITR) sequence, a promoter sequence, a nucleic acid sequence represented by SEQ ID NO:15, which encodes a codon-optimized ATP7B Δ1-3-SS, and an AAV 3′-inverted terminal repeat sequence (ITR). In some exemplary embodiments, the present disclosure provides a vector genome comprising of SEQ ID NO:14, which comprises an AAV 5′-inverted terminal repeat (ITR) sequence, a promoter sequence, a nucleic acid sequence represented by SEQ ID NO:1, which encodes native ATP7B Δ1-3-SS, or an adeno-associated virus (AAV) vector comprising the same.

In additional aspects, the application provides recombinant nucleic acid sequences corresponding to vector genomes useful in the treatment of WD. In some embodiments, the application provides a recombinant nucleic acid which is 80%, 85%, 90%, 91%, 92%, 93%, 94%, 95%, 96%, 97%, 98%, 99% or more identical to SEQ ID NO:14. Thus, the application provides recombinant nucleic acids which are at least 80% (e.g., 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) identical to SEQ ID NO:14. In an exemplary embodiment, the application provides a recombinant nucleic acid sequence corresponding to a vector genome comprising an AAV 5′-inverted terminal repeat (ITR) sequence, a promoter sequence, a nucleic acid sequence represented by SEQ ID NO:1, which encodes native ATP7B Δ1-3-SS, and an AAV 3′-inverted terminal repeat sequence (ITR), wherein the vector genome comprises or consists of SEQ ID NO:14. In an exemplary embodiment, the application provides a recombinant nucleic acid sequence corresponding to a vector genome comprising an AAV 5′-inverted terminal repeat (ITR) sequence, a promoter sequence, a nucleic acid sequence represented by SEQ ID NO:15, which encodes a codon-optimized ATP7B Δ1-3-SS, and an AAV 3′-inverted terminal repeat sequence (ITR).

### Promoter:

In various aspects described herein, AAV vectors are provided which comprises a promoter sequence which helps drive and regulate transgene expression, e.g., expression of ATP7B Δ1-3-SS (e.g., amino acid sequence of ATP7B Δ1-3-SS represented by SEQ ID NO:8). In exemplary embodiments, the promoter sequence is located between the selected 5′-ITR sequence and the coding sequence for ATP7B Δ1-3-SS (e.g., SEQ ID NO:1 or SEQ ID NO:15). In some embodiments, the promoter sequence is located downstream of an enhancer sequence. In some embodiments the promoter sequence is located upstream of an intron sequence. In some illustrative embodiments, a vector described herein uses the transthyretin (TTR) promoter, which may optionally be located downstream of a transthyretin enhancer (enTTR).

In some embodiments, the promoter is selected from a transthyretin (TTR) promoter, a chicken j-actin (CBA) promoter, a cytomegalovirus immediate early gene (CMV) promoter, a thyroxine binding globulin (TBG) promoter, an alpha-1 anti-trypsin (A1AT) promoter, and a CAG promoter (constructed using the CMV early enhancer element, the promoter, the first exon, and the first intron of CBA gene, and the splice acceptor of the rabbit beta-globin gene). In an exemplary embodiment, the promoter is the TTR promoter. In one embodiment, the TTR promoter comprises or consists of SEQ ID NO:12.

In addition to a promoter, an AAV vector may contain other appropriate transcription initiation, termination, enhancer sequence, and efficient RNA processing signals. As described in further detail below, such sequences include splicing and polyadenylation (poly A) signals, regulatory elements that enhance expression (i.e., WPRE), sequences that stabilize cytoplasmic mRNA, sequences that enhance translation efficiency (i.e., the Kozak consensus sequence), and sequences that enhance protein stability.

In some embodiments, the AAV vector contains a vector genome that further comprises a consensus Kozak sequence. In some embodiments, the consensus Kozak sequence is located downstream of an intron sequence. In one embodiment, the consensus Kozak sequence is GCCGCC. As will be understood by those skilled in the art, the consensus Kozak sequence is typically located immediately upstream of a coding sequence; in this case, immediately upstream of a coding sequence for truncated ATP7B (ATP7B Δ1-3-SS) (e.g., SEQ ID NO:1 or SEQ ID NO:15). As will be appreciated by the skilled artisan, the consensus Kozak sequence can be considered to share an ATG residue corresponding to the start codon of the therapeutic polypeptide, e.g., truncated ATP7B (ATP7B Δ1-3-SS) (e.g., SEQ ID NO:1 or SEQ ID NO:15). For the simplicity of disclosure, the consensus Kozak sequence, as described herein, comprises a six-nucleotide sequence corresponding to the region not shared with the nucleic acid encoding the therapeutic polypeptide, e.g., truncated ATP7B (ATP7B Δ1-3-SS encoded by SEQ ID NO:1 or SEQ ID NO:15).

### ATP7B Polypeptides:

As described herein, aspects of the invention provide recombinant vectors that include a genome that comprises an AAV 5′-inverted terminal repeat sequence (ITR), a promoter sequence, a coding sequence for truncated human ATP7B (ATP7B Δ1-3-SS) having an amino acid sequence of SEQ ID NO:8 (e.g., SEQ ID NO:1 or SEQ ID NO:15), and an AAV 3′-inverted terminal repeat sequence (ITR). ATP7B has eight transmembrane domains that form a path through cell membranes for copper translocation; and a large N-terminus with six metal-binding domains (MBDs), each comprising approximately 70 amino acids and the highly conserved metal-binding motif GMxCxxC (where x is any amino acid). In addition to the canonical sequence (also called isoform a, which is the longest isoform; NCBI Reference Sequence: NP_000044.2), four additional isoforms are known: NCBI Reference Sequences four additional isoforms are known: NCBI Reference Sequences NP_001005918.1, NP_001230111.1, NP_001317507.1, NP_001317 508.1. The compositions and methods described herein may be used to treat subjects having a non-functional ATP7B variant protein which causes disease.

In one embodiment, a coding sequence for truncated human ATP7B (ATP7B Δ1-3-SS) (e.g., SEQ ID NO:1 or SEQ ID NO:15) encodes for a protein with amino acids as described in SEQ ID NO:8. SEQ ID NO:1 provides the cDNA for native human ATP7B that has MBDs 1-3 deleted. SEQ ID NO: 8 represents DEL1-3 Native or ATP7B Δ1-3-SS protein, in which two serine residues corresponding to positions 340 and 341 of the wild-type ATP7B full-length protein sequence are present.

In various embodiments described herein, vectors are provided that contain a genome comprising a coding sequence for truncated ATP7B (ATP7B Δ1-3-SS) (e.g., SEQ ID NO:1 or SEQ ID NO:15).

In some embodiments, vectors are provided that contain a genome comprising an engineered cDNA for human ATP7B, which has been codon-optimized (e.g., SEQ ID NO:15). The polypeptides delivered with the vectors described herein encompass truncated ATP7B in which MBDs 1-3 have been deleted (ATP7B Δ1-3-SS), which are suitable for use in treating WD.

In some embodiments, the polypeptide expressed with a vector described herein is a truncated human ATP7B (SEQ ID NO:8).

### Vector Elements:

In some embodiments, the AAV vector contains a genome that further comprises one or more enhancer sequences. In one embodiment, the enhancer is selected from a transthyretin enhancer (enTTR), a cytomegalovirus immediate early gene (CMV) enhancer, a chicken 3-actin (CBA) enhancer, an En34 enhancer, and an apolipoprotein (ApoE) enhancer. In an exemplary embodiment, the enhancer is the enTTR enhancer. In one embodiment, the enTTR enhancer comprises or consists of SEQ ID NO:3.

In some embodiments, the AAV vector contains a genome that further comprises one or more intron sequences. In one embodiment, the intron is selected from an SV40 Small T intron, a rabbit hemoglobin subunit beta (rHBB) intron, a human beta globin IVS2 intron, a β-globin/IgG chimeric intron (Promega chimeric intron), or an hFIX intron. In one exemplary embodiment, the intron is the SV40 Small T intron. In one embodiment, the SV40 Small T intron sequence comprises or consists of SEQ ID NO:4. In another exemplary embodiment, the intron is the rHBB intron. In one embodiment, the rHBB intron sequence comprises or consists of SEQ ID NO:5.

In some embodiments, the AAV vector contains a genome that further comprises a polyadenylation signal sequence. In one embodiment, the polyadenylation signal sequence is selected from an SV40 polyadenylation signal sequence, a bovine growth hormone (BGH) polyadenylation signal sequence, and a rabbit beta globin polyadenylation signal sequence. In an exemplary embodiment, the polyadenylation signal sequence is the bovine growth hormone (BGH) polyadenylation signal sequence. In one embodiment, the BGH polyadenylation signal sequence comprises or consists of SEQ ID NO:6. In another exemplary embodiment, the polyadenylation signal sequence is the SV40 polyadenylation signal sequence. In one embodiment, the SV40 polyadenylation signal sequence comprises or consists of SEQ ID NO:7.

### AAV Capsids:

In another aspect, the application provides recombinant adeno-associated virus (rAAV) useful as agents for gene therapy in the treatment of WD, wherein said rAAV comprises an AAV capsid, and a vector genome as described herein. In some embodiments, the AAV capsid is from an AAV of serotype 9, 8, 1, 2, 3, 4, 5, 6, 7, 10, 11, 12, rh10, or hu37 (i.e., AAV1, AAV2, AAV3, AAV4, AAV5, AAV6, AAV7, AAV8, AAV9, AAV10, AAV11, AAV12, AAVrh10, or AAVhu37). In an exemplary embodiment, the AAV vector is an AAV serotype 9 (AAV9) vector, an AAV9 variant vector, an AAV serotype 8 (AAV8) vector, an AAV serotype 5 (AAV5) vector, or an AAV serotype 2 (AAV2) vector. In certain embodiments, the AAV capsid and vector are from an AAV9 serotype. In certain embodiments, the AAV capsid and vector are from an AAV8 serotype.

The AAV9 capsid is a self-assembled AAV capsid composed of multiple AAV9 VP proteins. The AAV9 VP proteins are typically expressed as alternative splice variants encoded by a nucleic acid sequence of SEQ ID NO:9 or a sequence that is at least 70%, at least 75%, at least 80%, at least 85%, at least 90%, at least 95%, at least 97%, at least 99% thereto, which encodes the capsid protein VP1 amino acid sequence of SEQ ID NO:10 (GenBank Accession: AAS99264). These splice variants result in proteins of different length of SEQ ID NO:10. In certain embodiments, an AAV9 capsid include an AAV9 capsid protein having an amino acid sequence which is 99% identical to AAS99264 or 99% identical to SEQ ID NO:10. See also U.S. Pat. No. 7,906,111, and international publication No. WO/2005/033321. As used herein, an AAV9 variant includes those described in, e.g., international publication No. WO/2016/049230, U.S. Pat. No. 8,927,514, U.S. Patent Publication No. 2015/0344911, and U.S. Pat. No. 8,734,809.

As indicated herein, the AAV9 capsid sequences and capsid proteins encoded by the sequences (e.g., nucleic acid sequence of SEQ ID NO:9 or amino acid sequence of SEQ ID NO:10 encoding AAV9 capsid protein VP1) are useful in the production of rAAV. However, in other embodiments, another AAV capsid is selected. Tissue specificity is determined by the capsid type. AAV serotypes which transduce a suitable target (e.g., liver, muscle, lung, or CNS) may be selected as sources for capsids of AAV viral vectors including, e.g., AAV1, AAV2, AAV3, AAV4, AAV5, AAV6, AAV6.2, AAV7, AAV8, AAV9, AAVrh10, AAVrh64R1, AAVrh64R2, AAVrh8. See, e.g., U.S. Patent Publication No. 2007/0036760; U.S. Patent Publication No. 2009/0197338; and EP1310571. See also international application No. WO 2003/042397 (AAV7 and other simian AAV), U.S. Pat. Nos. 7,282,199 and 7,790,449 (AAV8). In addition, AAV yet to be discovered, or a recombinant AAV based thereon, may be used as a source for the AAV capsid. These documents also describe other AAV which may be selected for generating AAV and are incorporated by reference. In some embodiments, an AAV capsid for use in the viral vector can be generated by mutagenesis (i.e., by insertions, deletions, or substitutions) of one of the aforementioned AAV capsids or its encoding nucleic acid.

### Host Cells Comprising a Recombinant Nucleic Acid Molecule:

In some aspects, provided herein are host cells comprising a recombinant nucleic acid molecule, viral vector, e.g., an AAV vector, or a rAAV disclosed herein. In specific embodiments, the host cells may be suitable for the propagation of AAV.

A vast range of host cells can be used, such as bacteria, yeast, insect, mammalian cells, etc. In some embodiments, the host cell can be a cell (or a cell line) appropriate for production of recombinant AAV (rAAV), for example, a HeLa, Cos-7, HEK293, A549, BHK, Vero, RD, HT-1080, ARPE-19, or MRC-5 cell. In certain embodiments, the host cell line of the present invention is a HeLa cell line (e.g., HeLa S3). In another embodiment, host cell line of the present invention is a HEK293 cell line.

The recombinant nucleic acid molecules or vectors can be delivered into the host cell culture using any suitable method known in the art. In some embodiments, a stable host cell line that has the recombinant nucleic acid molecule or vector inserted into its genome is generated. In some embodiments, a stable host cell line is generated, which contains an AAV vector described herein. After transfection of the AAV vector to the host culture, integration of the rAAV into the host genome can be assayed by various methods, such as antibiotic selection, fluorescence-activated cell sorting, southern blot, PCR based detection, fluorescence in situ hybridization as described by Nakai et al., Nature Genetics (2003) 34:297-302; Philpott et al., Journal of Virology (2002) 76(11):5411-5421, and Howden et al., J. Gene Med. (2008) 10:42-50. Furthermore, a stable cell line can be established according to protocols well known in the art, such as those described in Clark, Kidney International Vol. 61 (2002):S9-S15, and Yuan et al., Human Gene Therapy (2011) 22(5):613-24.

### Recombinant AAV for Gene Therapy:

Adeno-associated virus (AAV) belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency.

The AAV genome is characterized by two inverted terminal repeats (ITRs) that flank two open reading frames (ORF). The AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Days and Berns, Clin. Microbiol. Rev. (2008) 21(4):583-593).

The left ORF of AAV contains the Rep gene, which encodes four proteins—Rep78, Rep68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin. Microbiol. Rev. (2008) 21(4):583-593).

AAV is currently one of the most frequently used viruses for gene therapy. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of AAV for the recombinant nucleic acid molecules and methods disclosed herein.

AAV possesses several desirable features for a gene therapy vector, including the ability to bind and enter target cells, enter the nucleus, the ability to be expressed in the nucleus for a prolonged period of time, and low toxicity. However, the small size of the AAV genome limits the size of heterologous DNA that can be incorporated. To minimize this problem, AAV vectors have been constructed that do not encode Rep and the integration efficiency element (IEE). The ITRs are retained as they are cis signals required for packaging (Daya and Berns, Clin. Microbiol. Rev. (2008) 21(4):583-593).

Methods for producing rAAV suitable for gene therapy are well known in the art (see, e.g., U.S. Patent Application Nos. 2012/0100606; 2012/0135515; 2011/0229971; and 2013/0072548; and Ghosh et al., Gene Ther. (2006) 13(4):321-329), and can be utilized with the recombinant nucleic acid molecules and methods disclosed herein.

In some aspects, the application relates to the use of an rAAV disclosed herein for the treatment of Wilson disease (WD), wherein the rAAV includes an AAV capsid and a vector genome packaged therein. In some embodiments, the vector contains a genome comprising as operably linked components in 5′ to 3′ order: a 5′-inverted terminal repeat sequence (ITR), a promoter sequence, a coding sequence for a truncated human ATP7B (ATP7B Δ1-3-SS) (e.g., SEQ ID NO:1 or SEQ ID NO:15), and a 3′-inverted terminal repeat sequence (ITR). In an exemplary embodiment, the vector genome also comprises an enhancer sequence upstream of the promoter sequence, an intron downstream of the promoter, and a polyadenylation sequence upstream of the 3′-ITR. Thus, in another exemplary embodiment, the vector genome comprises as operably linked components in 5′ to 3′ order: a 5′-inverted terminal repeat sequence (ITR), an enhancer sequence, a promoter sequence, an intron sequence, a coding sequence for a truncated human ATP7B (ATP7B Δ1-3-SS) (e.g., SEQ ID NO:1 or SEQ ID NO:15), a polyadenylation signal sequence, and a 3′-inverted terminal repeat sequence (ITR). In a further exemplary embodiment, the vector genome comprises as operably linked components in 5′ to 3′ order: an AAV2 5′-ITR sequence, an enTTR enhancer, a TTR promoter, an SV40 Small T intron, a coding sequence for a truncated human ATP7B (ATP7B Δ1-3-SS) (e.g., SEQ ID NO:1 or SEQ ID NO:15), an SV40 polyadenylation signal sequence, and an AAV2 3′-ITR. In some embodiments, the vector genome further comprises a consensus Kozak sequence located downstream of the intron sequence. In some embodiments, the capsid is an AAV9 capsid.

In some aspects, the application relates to the use of a rAAV disclosed herein for the treatment of Wilson disease (WD), wherein the rAAV includes an AAV capsid and a vector genome packaged therein. In some embodiments, the vector genome comprises as operably linked components in 5′ to 3′ order: a 5′-inverted terminal repeat sequence (ITR), a promoter sequence, a coding sequence for a truncated human ATP7B (ATP7B Δ1-3-SS) (e.g., SEQ ID NO:1), and a 3′-inverted terminal repeat sequence (ITR). In an exemplary embodiment, the vector genome also comprises an enhancer sequence upstream of the promoter sequence, an intron downstream of the promoter, and a polyadenylation sequence upstream of the 3′-ITR. Thus, in another exemplary embodiment, the vector contains a genome comprising as operably linked components in 5′ to 3′ order: a 5′-inverted terminal repeat sequence (ITR), an enhancer sequence, a promoter sequence, an intron sequence, a coding sequence for a truncated human ATP7B (ATP7B Δ1-3-SS) (e.g., SEQ ID NO:1), a polyadenylation signal sequence, and a 3′-inverted terminal repeat sequence (ITR). In a further exemplary embodiment, the vector contains a genome comprising as operably linked components in 5′ to 3′ order: an AAV2 5′-ITR sequence, an enTTR enhancer, a TTR promoter, an SV40 Small T intron, a coding sequence for a truncated human ATP7B (ATP7B Δ1-3-SS) (e.g., SEQ ID NO:1), an SV40 polyadenylation signal sequence, and an AAV2 3′-ITR. In some embodiments, the packaged genome further comprises a consensus Kozak sequence located downstream of the intron sequence. In some embodiments, the capsid is an AAV9 capsid.

In some aspects, the application relates to the use of an rAAV disclosed herein for the treatment of Wilson disease (WD), wherein the rAAV includes an AAV capsid and a packaged vector genome. In some embodiments, the vector contains a packaged genome comprising as operably linked components in 5′ to 3′ order: a 5′-inverted terminal repeat sequence (ITR), a promoter sequence, a coding sequence for a truncated human ATP7B (ATP7B Δ1-3-SS) (e.g., SEQ ID NO:15), and a 3′-inverted terminal repeat sequence (ITR). In an exemplary embodiment, the packaged genome also comprises an enhancer sequence upstream of the promoter sequence, an intron downstream of the promoter, and a polyadenylation sequence upstream of the 3′-ITR. Thus, in another exemplary embodiment, the vector contains a packaged genome comprising as operably linked components in 5′ to 3′ order: a 5′-inverted terminal repeat sequence (ITR), an enhancer sequence, a promoter sequence, an intron sequence, a coding sequence for a truncated human ATP7B (ATP7B Δ1-3-SS) (e.g., SEQ ID NO:15), a polyadenylation signal sequence, and a 3′-inverted terminal repeat sequence (ITR). In a further exemplary embodiment, the vector contains a packaged genome comprising as operably linked components in 5′ to 3′ order: an AAV2 5′-ITR sequence, an enTTR enhancer, a TTR promoter, an SV40 Small T intron, a coding sequence for a truncated human ATP7B (ATP7B Δ1-3-SS) (e.g., SEQ ID NO:15), an SV40 polyadenylation signal sequence, and an AAV2 3′-ITR. In some embodiments, the packaged genome further comprises a consensus Kozak sequence located downstream of the intron sequence. In some embodiments, the capsid is an AAV9 capsid.

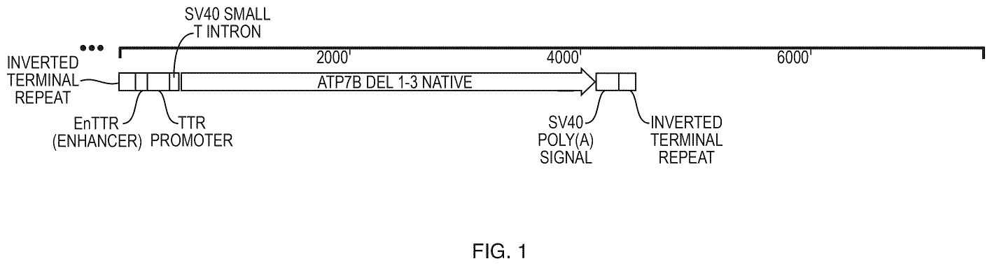

An illustrative diagram showing an exemplary packaged vector genome construct for the expression of truncated ATP7B retaining MBDs 4, 5, and 6 is provided in FIG. 1. A 5′-ITR is represented by nucleotides 1-145; an enTTR enhancer is represented by nucleotides 146-245; a TTR promoter is represented by nucleotides 246-435; an SV40 Small T intron is represented by nucleotides 436-530; a consensus Kozak sequence is represented by nucleotides 531-536; a truncated ATP7B coding sequence is represented by nucleotides 540-4142; an SV40 polyadenylation signal sequence is represented by nucleotides 4143-4340; and a 3′-ITR is represented by nucleotides 4341-4485.

In certain embodiments, the nucleic acid sequence encoding ATP7B Δ1-3-SS is the native human sequence (represented by SEQ ID NO:1). Alternatively, in some embodiments, the nucleic acid sequence encoding ATP7B Δ1-3-SS is a codon-optimized human sequence (represented by SEQ ID NO:15).

### Improved Efficacy in Treating WD:

In certain embodiments, the truncated human ATP7B (ATP7B Δ1-3-SS) encoded by SEQ ID NO:1 or SEQ ID NO:15, described herein, is more efficacious than the full-length or other truncated forms of ATP7B (e.g., ATP7B Δ1-4, SEQ ID NO:13). In some aspects, the ATP7B Δ1-3-SS of the present disclosure localizes to the Trans Golgi Network (TGN). In certain embodiments, a rAAV comprising the nucleic acid sequence of SEQ ID NO:1, which encodes ATP7B Δ1-3-SS, upon injection to a mammal diagnosed with a disorder of copper metabolism (e.g., Wilson disease), decreases the copper levels in the liver and urine of the mammal.

### Improved Yield of AAV Vectors Comprising Truncated ATP7B:

In one aspect, the rAAV comprising the nucleic acid sequence encoding ATP7B Δ1-3-SS, packaged in AAV8 or AAV9, described herein, has about 1.1-about 10-fold higher (e.g., about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 3-fold, about 4-fold, about 5-fold, about 6-fold, about 7-fold, about 8-fold, about 9-fold, or about 10-fold) manufacturing yield than that of full-length ATP7B or the ATP7B Δ1-4.

### Improved Yield of AAV Vector Comprising AAV9 Capsid:

In one aspect, the rAAV comprising the AAV9 capsid has about 1.1-about 10 fold higher (e.g., about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 3-fold, about 4-fold, about 5-fold, about 6-fold, about 7-fold, about 8-fold, about 9-fold, or about 10-fold) titer yield in comparison to rAAV comprising the AAV8 capsid.

### Pharmaceutical Compositions:

Compositions comprising the rAAV disclosed herein and a pharmaceutically acceptable carrier are provided by the present disclosure. Suitable pharmaceutical formulations for administration of rAAV can be found, for example, in U.S. Patent Application Publication No. 2012/0219528. The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

As highlighted in the preceding paragraph, the application relates in some aspects to pharmaceutical compositions comprising a rAAV of the invention. In some embodiments, the pharmaceutical composition comprises a pharmaceutically acceptable carrier or excipient. In some embodiments, the pharmaceutical composition is formulated for subcutaneous, intramuscular, intradermal, intraperitoneal, or intravenous administration. In an exemplary embodiment, the pharmaceutical composition is formulated for intravenous administration.

In some embodiments, the rAAV is formulated in a buffer/carrier suitable for infusion in human subjects. The buffer/carrier should include a component that prevents the rAAV from sticking to the infusion tubing but does not interfere with the rAAV binding activity in vivo. Various suitable solutions may include one or more of: a buffering saline, a surfactant, and a physiologically compatible salt or mixture of salts adjusted to an ionic strength equivalent to about 100 mM sodium chloride (NaCl) to about 250 mM NaCl, or a physiologically compatible salt adjusted to an equivalent ionic concentration. The pH may be in the range of 6.5 to 8.5, or 7 to 8.5, or 7.5 to 8. A suitable surfactant, or combination of surfactants, may be selected from among Poloxamers, i.e., nonionic triblock copolymers composed of a central hydrophobic chain of polyoxypropylene 10 (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol.

### Methods of Treating Wilson Disease:

In yet another aspect, the application relates to methods of treating WD in a human subject comprising administering to the human subject a therapeutically effective amount of a rAAV including SEQ ID NO:1 or SEQ ID NO:15 for encoding a truncated ATP7B (ATP7B Δ1-3-SS), disclosed herein.

In one embodiment, the application provides a method of treating WD comprising administering a rAAV that includes an AAV capsid and a packaged vector genome, wherein the vector genome comprises a coding sequence for a truncated human ATP7B (ATP7B Δ1-3-SS) (e.g., SEQ ID NO:1 or SEQ ID NO:15).

In yet another aspect, the application relates to methods of treating WD in a human subject comprising administering to a human subject diagnosed with at least one mutation in ATP7B, a therapeutically effective amount of at least one rAAV comprising a vector genome comprising a coding sequence for a truncated human ATP7B (ATP7B Δ1-3-SS) (e.g., SEQ ID NO:1 or SEQ ID NO:15). In one embodiment, the application provides a method of treating WD in a human subject diagnosed with at least one mutation in ATP7B comprising administering a rAAV that includes an AAV capsid and a packaged vector genome, wherein the vector genome comprises coding sequence for a truncated human ATP7B (ATP7B Δ1-3-SS) (e.g., SEQ ID NO:1 or SEQ ID NO:15). The coding sequence as represented by SEQ ID NO:1 encodes a truncated ATP7B represented by SEQ ID NO:8. In some embodiments, the capsid is an AAV9 capsid.

In yet another aspect, the application relates to methods of treating WD in a human subject comprising administering to a human subject diagnosed with at least one mutation in ATP7B, a therapeutically effective amount of at least one rAAV comprising a vector genome comprising a coding sequence for a truncated human ATP7B (ATP7B Δ1-3-SS) (e.g., SEQ ID NO:1). In one embodiment, the application provides a method of treating WD in a human subject diagnosed with at least one mutation in ATP7B comprising administering a rAAV that includes an AAV capsid and a packaged vector genome, wherein the vector genome comprises coding sequence for a truncated human ATP7B (ATP7B Δ1-3-SS) (e.g., SEQ ID NO:1). The coding sequence as represented by SEQ ID NO:1 encodes a truncated ATP7B represented by SEQ ID NO:8. In some embodiments, the capsid is an AAV9 capsid.

In yet another aspect, the application relates to methods of treating WD in a human subject comprising administering to a human subject diagnosed with at least one mutation in ATP7B, a therapeutically effective amount of at least one rAAV comprising a vector genome comprising a coding sequence for a truncated human ATP7B (ATP7B Δ1-3-SS) (e.g., SEQ ID NO:15). In one embodiment, the application provides a method of treating WD in a human subject diagnosed with at least one mutation in ATP7B comprising administering a rAAV that includes an AAV capsid and a packaged vector genome, wherein the vector genome comprises coding sequence for a truncated human ATP7B (ATP7B Δ1-3-SS) (e.g., SEQ ID NO:15). In some embodiments, the capsid is an AAV9 capsid.

Any suitable method or route can be used to administer a rAAV or a rAAV-containing composition described herein. Routes of administration include, for example, systemic, oral, inhalation, intranasal, intratracheal, intraarterial, intraocular, intravenous, intramuscular, subcutaneous, intradermal, and other parenteral routes of administration. In some embodiments, the rAAV or a composition comprising a rAAV is administered intravenously.

The specific dose administered can be a uniform dose for each patient, for example, 1.0×10¹¹-1.0×10¹⁴ viral genome per kilogram of patient body weight (vg)/kg. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can also be adjusted as the progress of the disease is monitored.

In some embodiments, the rAAV is administered at a dose of, e.g., about 1.0×10¹¹ vg/kg to about 1×10¹⁴ vg/kg, about 5×10¹¹ vg/kg to about 5×10¹³ vg/kg, or about 1×10¹² to about 1×10¹³ vg/kg, as measured by qPCR or digital droplet PCR (ddPCR). In some embodiments, the rAAV is administered at a dose of about 2×10¹² vg/kg. In some embodiments, the rAAV is administered at a dose of about 5×10¹² vg/kg. In some embodiments, the rAAV is administered at a dose of about 6×10¹² vg/kg. In some embodiments, the rAAV is administered at a dose of about 1×10¹³ vg/kg. In some embodiments, the rAAV is administered at a dose of about 7×10¹³ vg/kg. The rAAV can be administered in a single dose, or in multiple doses (such as 2, 3, 4, 5, 6, 7, 8, 9, 10 or more doses) as needed for the desired therapeutic results. In some exemplary embodiments, only a single dose of a particular rAAV is administered.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including” is intended merely to illustrate better the present invention and does not pose a limitation on the scope of the invention unless claimed. No language in the specification should be construed as indicating any non-claimed element as essential to the practice of the present invention.

### EXAMPLES

The invention now being generally described, will be more readily understood by reference to the following examples, which are included merely for purposes of illustration of certain aspects and embodiments of the present invention, and is not intended to limit the invention.

### Example 1—AAV Vectors and rAAV Produced from the Vectors

### AAV Vector

This example describes construction of an AAV vector with the nucleic acid sequence represented by SEQ ID NO:1, bounded by two AAV2 inverted terminal repeats (ITRs, SEQ ID NO:2). SEQ ID NO:1 represents the cDNA for native human ATP7B that has MBDs 1-3 deleted. Nucleotides 223-225 in SEQ ID NO:1 encode for serine residue, S340 and nucleotides 226-228 in SEQ ID NO:1 encode for serine residue, S341 (numbering based on wild-type full length ATP7B protein sequence).

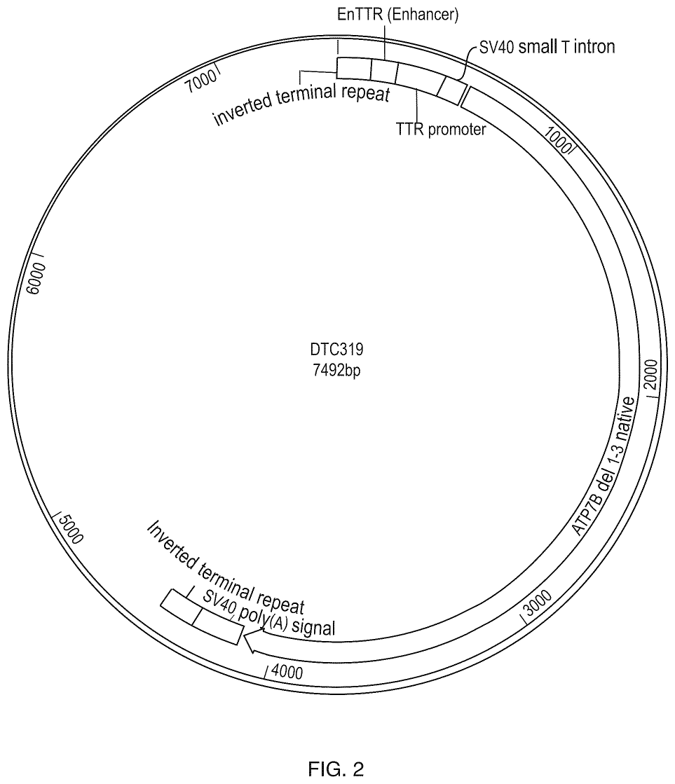

As illustrated in FIG. 1, within the AAV vector the ATP7B expression cassette contains an enhancer (EnTTR), a promoter (TTR), an intron (SV40 small T intron), the nucleotide sequence of SEQ ID NO:1, which encodes a truncated human ATP7B (ATP7B Δ1-3-SS), and an SV40 poly (A) signal. A circular map of the vector illustrating various components is shown in FIG. 2.

The AAV vector DTC319 contains a truncated human ATP7B sequence in which metal-binding domains 4, 5, and 6 are retained. The truncated human ATP7B sequence encodes for a protein that comprises two serine residues S340 and S341 (numbered according to NCBI Reference Sequence: NP_000044.2) represented by SEQ ID NO:8.

The Simian virus 40 (SV40) late polyadenylation signal (Genbank Accession No. J02400 (SEQ ID NO:7) provides a cis sequence for efficient polyadenylation of the ATP7B mRNA. This element functions as a signal for a specific cleavage event at the 3′-end of the nascent transcript and addition of a long polyadenyl tail.

Each truncated ATP7B expression cassette was cloned into an AAV vector. All AAV vectors had a backbone encoding the kanamycin-resistance gene. An exemplary AAV vector DTC319 is illustrated in FIG. 2. FIG. 1 depicts the expression cassette of DTC319, for expressing ATP7B (ATP7B Δ1-3-SS).

rAAV Virions

The AAV vector genome is a single-stranded DNA genome. Only the sequences between and inclusive of the ITR sequences are packaged into the AAV virion. Virions were produced by transfection of three plasmids into human embryonic kidney 293 (HEK293) cells, which provide E1a and E1b gene products. The first plasmid can be an AAV vector described herein. The second plasmid may be a packaging plasmid containing the wild-type AAV2 rep and AAV8 or AAV9 cap genes. The third plasmid is a helper adenovirus plasmid.

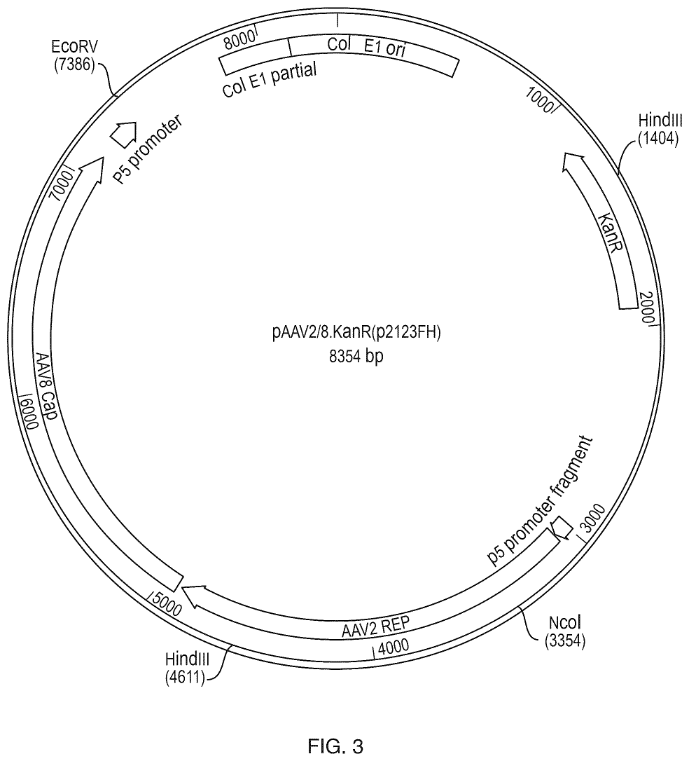

Illustration of an exemplary packaging plasmid, pAAV2/8.KanR (p2123FH) plasmid, is shown in FIG. 3. In this plasmid, the adeno-associated Rep/Cap plasmid pAAV2/8.KanR(p2123FH) (8354 bp) encodes the four wild-type AAV2 viral replication (Rep) proteins and the three wild-type AAV VP capsid (cap) proteins from serotype 8. Within the plasmid, the AAV p5 promoter that normally drives Rep gene expression has been moved from the 5′-end of the Rep region to the 3′-end of the AAV8 cap region. This arrangement introduces a spacer between the promoter and the Rep gene (i.e., the plasmid backbone) resulting in down-regulation of the expression of Rep and an increase in the ability to support high titer rAAV production. The gene for kanamycin resistance and the MB1 origin are included for plasmid production in E. coli.

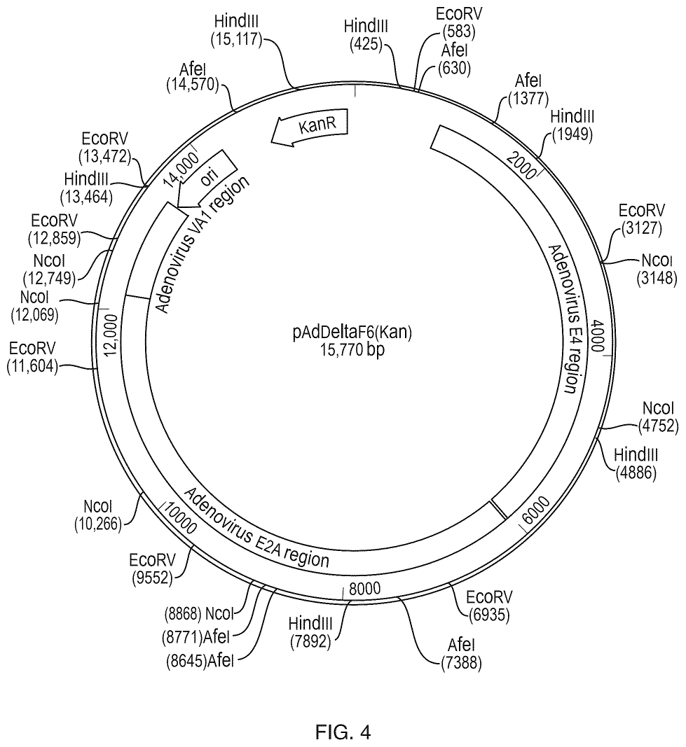

Illustration of an exemplary helper plasmid, pAdDeltaF6 (Kan), is shown in FIG. 4. In this plasmid, regions of the adenovirus genome that are important for AAV replication, namely E2A, E4, and VA RNA, are provided. The adenovirus E1 functions are also required but are provided by the HEK293 host cells. The plasmid shown in FIG. 4 does not contain other adenovirus replication, structural genes, or the cis elements critical for adenovirus replication such as the adenoviral ITRs and therefore, no infectious adenovirus is expected to be generated. The gene for kanamycin resistance and the MB1 origin are included for plasmid production in E. coli.

### Example 2—Deletion of Metal-Binding Domains (MBDs) 1-3 in Human ATP7B Improves Manufacturing Yield

This example describes experiments, which demonstrated that ATP7B Δ1-3-SS had higher yield than the full-length ATP7B or the truncated form ATP7B Δ1-4.

Lack of functional ATP7B results in accumulation of copper in the liver and other tissues, which manifests as liver disease with neurological or psychiatric symptoms. WD can be treated by reducing copper absorption or removing excess copper from the body. C3He-Atp7b^{tx-j} mice do not express functional Atp7b and thus serve as a mouse model for WD. AAV vectors containing the codon-optimized full-length human ATP7B sequence were used to transfect HEK293 cells with the Rep/Cap plasmid, which encodes four wild-type AAV2 viral replication (Rep) proteins and the three wild-type AAV VP capsid (cap) proteins, from serotype 8 and the helper plasmid to obtain ATP7BcoFL virus particles.

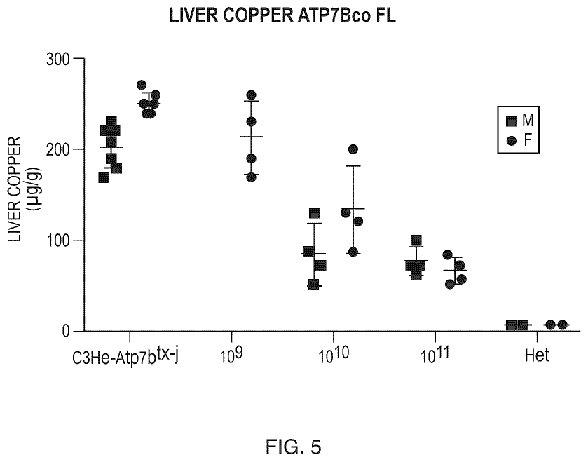

Male C3He-Atp7b^{tx-j} mice were intravenously (i.v.) injected with either 10¹⁰ or 10¹¹ GC/kg of ATP7BcoFL (full-length human ATP7B which has been codon-optimized). Female C3He-Atp7b^{tx-j} mice were injected i.v. with either 10⁹, 10¹⁰, or 10¹¹ GC/kg of the same vector. Liver copper levels in males (denoted by squares) and females (denoted by circles) were evaluated by inductively coupled plasma-mass spectrometry (ICP-MS), and compared to the copper levels from age-matched uninjected male and female heterozygous (Het) and C3He-Atp7b^{tx-j} mice. Mice were necropsied at approximately 9 months of age and liver was harvested. Data is presented in FIG. 5.

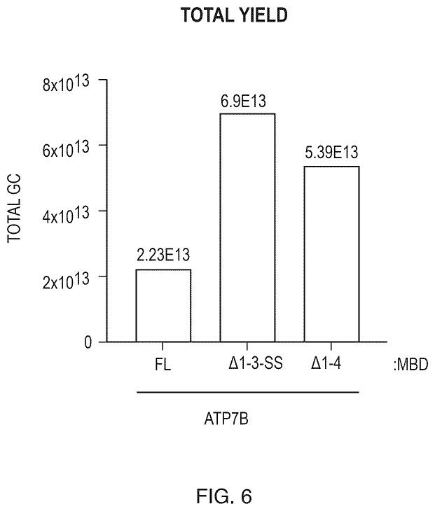

Gene therapy utilizing AAV vectors can be used for treating WD. However, there is a limit to the size of the cDNA that can be packaged inside an AAV vector capsid. The wild type AAV genome is 4.7 kb, and packaging larger genomes can potentially reduce the yield and integrity of the DNA sequence encapsulated within the AAV capsid. Therefore, nucleotide sequence encoding ATP7B Δ1-3-SS was packaged within the AAV8 capsid, and manufacturing yield of ATP7B Δ1-3-SS was tested. AAV vectors encoding either full-length (FL) human ATP7B, human ATP7B in which MBDs 1-3 have been deleted, but the serine-rich loop including two serine residues (S340 and S341) between MBD3 and MBD4 is present (ATP7B Δ1-3-SS), or human ATP7B in which MBDs 1-4 have been deleted (ATP7B Δ1-4) were transfected into HEK293 cells. The Rep/Cap plasmid, which encodes four wild-type AAV2 viral replication (Rep) proteins and the three wild-type AAV VP capsid (cap) proteins from serotype 8, and the helper plasmid were co-transfected with the AAV vectors expressing various ATP7B proteins. FIG. 6 is a bar graph showing titers of rAAV produced from host cells after transfection of various AAV vectors. Y axis indicates total yield of each rAAV titer in genome copies (GC). The data show that ATP7B Δ1-3-SS had higher yield than the full-length or the ATP7B Δ1-4 truncated form.

### Example 3—ATP7B Δ1-3-SS is More Efficacious in Restoration of Copper Metabolism in Comparison to ATP7B FL

This example describes an experiment, which demonstrated that ATP7B Δ1-3-SS was more efficacious than ATP7B full-length (ATP7B FL) or ATP7B Δ1-4, in restoring copper metabolism in C3He-Atp7b^{tx-j} mice.

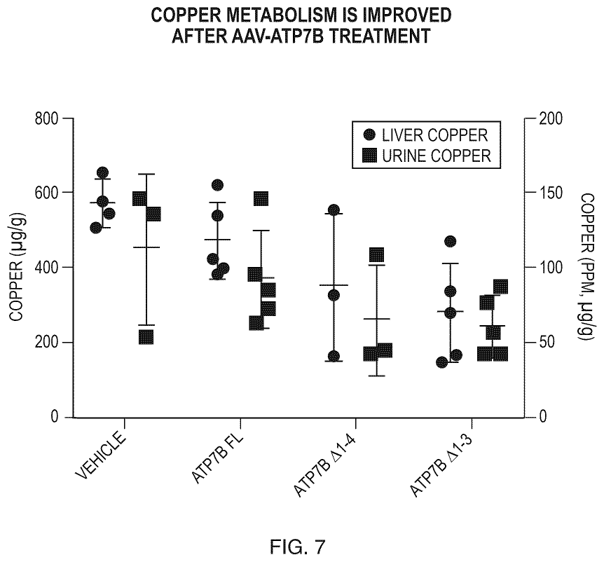

As described above in Example 2, packaging a bulky cDNA sequence inside an AAV vector capsid can reduce the integrity of the DNA sequence and have potential quality issues. Therefore, truncated versions of human ATP7B were packaged within the AAV8 capsid and their efficacy in restoring copper metabolism was tested. 1.0×10¹³ GC/kg of AAV8 vector encompassing either full length or truncated human ATP7B were administered to C3He-Atp7b^{tx-j} mice. Liver and urine copper levels were evaluated by inductively coupled plasma-mass spectrometry. FIG. 7 is a scatter plot of urine and liver copper levels, squares and circles, respectively (μg/g), assayed after C3He-Atp7b^{tx-j} mice were injected with AAV8 carrying full-length human ATP7B (ATP7B FL), ATP7B Δ1-3-SS, or ATP7B Δ1-4. FIG. 7 shows that ATP7B Δ1-3-SS is more efficacious than ATP7B full-length (ATP7B FL) or ATP7B Δ1-4, in restoring copper metabolism in C3He-Atp7b^{tx-j} mice. Phosphate-buffered saline (PBS) administered C3He-Atp7b^{tx-j} mice served as controls (vehicle).

### Example 4—AAV Vector Comprising AAV9 Capsid Showed Higher Viral Production

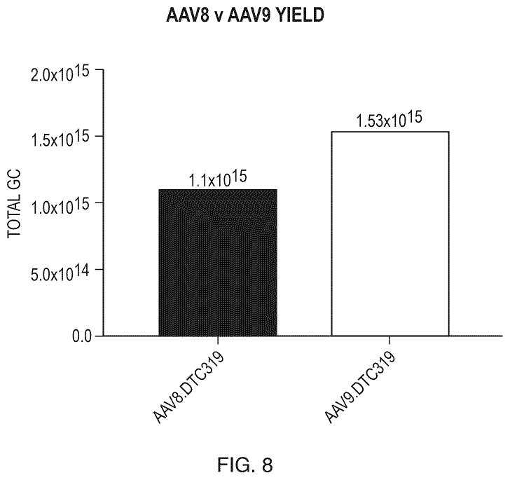

This example describes an experiment which showed that production of an AAV vector comprising the AAV9 capsid generates a higher yield in comparison to the production of an AAV vector comprising the AAV8 capsid. Different AAV vectors were titrated by qPCR quantifying DNase-resistant particles (DRPs). FIG. 8 shows total yield of rAAV (titers in genome copies (GC)) produced from host cells after transfection of an AAV vector (DTC319) that encodes a truncated human ATP7B, in which the metal-binding domains (MBDs) 1-3 have been deleted, but the serine-rich loop including two serine residues (S340 and S341) between MBD3 and MBD4 is present, and co-transfected with a plasmid encoding either AAV8 capsid or AAV9 capsid.

### Example 5—Therapeutic Properties of ATP7B Δ1-3-SS

This example describes animal studies, which demonstrated efficacy of ATP7B Δ1-3-SS (for example, DTC319, an rAAV vector that encodes a truncated human ATP7B, wherein metal-binding domains (MBDs) 1-3 have been deleted, but the serine-rich loop including two serine residues (S340 and S341) between MBD3 and MBD4 is present) in ameliorating symptoms of and treating Wilson disease (WD) in a mouse model (C3He-Atp7b^{tx-j}). In this example, three groups of male mice were evaluated: WD mice (C3He-Atp7b^{tx-j} mice) administered either an AAV infusion of ATP7B Δ1-3-SS (for example, DTC319) encoded in an AAV8 vector or an intravenous injection of vehicle control (dilution buffer), and wild-type (WT) mice, which served as a negative control. For the infusion, rAAV was produced by triple transient transfection of adherent HEK cells and purified by Cesium Chloride gradient ultracentrifugation, a purification method well-known in the art. At the study endpoint, 4 weeks after infusion, mice from each group were evaluated for liver copper accumulation, ceruloplasmin activity, and liver pathology.

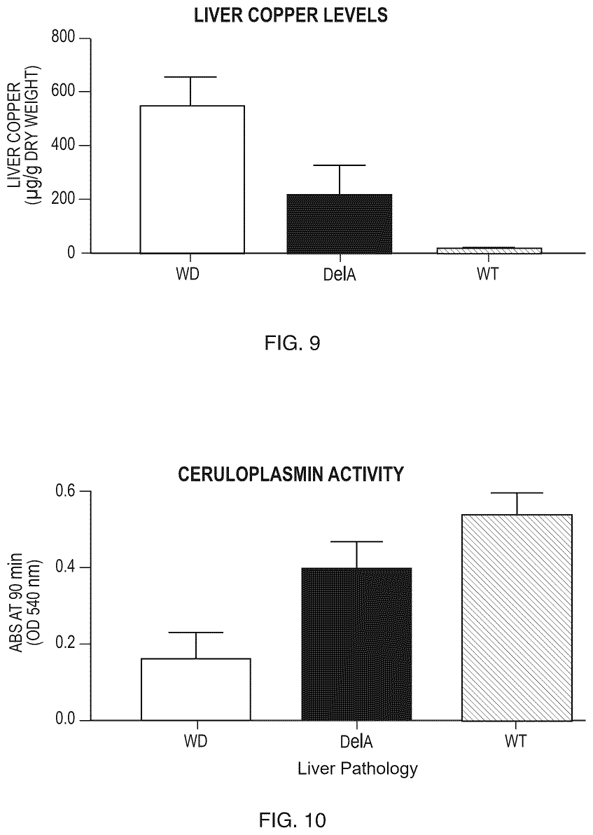

Liver copper accumulation was measured by inductively coupled plasma-mass spectrometry (ICP-MS), and demonstrated that liver copper level was significantly reduced in WD mice administered ATP7B Δ1-3-SS (for example, DTC319) (see FIG. 9, bar for DelA), compared to vehicle control. FIG. 9 shows liver copper accumulation levels (μg/g) in C3He-Atp7b^{tx-j} mice after being administered an intravenous injection of a vehicle control (dilution buffer, bar for WD) or an infusion of AAV8 carrying native ATP7B Δ1-3-SS (bar for DelA). Liver copper accumulation levels in uninjected wild-type mice (WT) represented in the bar graph served as a negative control. Values expressed as mean±SEM.

Ceruloplasmin activity was significantly increased in WD mice after being administered ATP7B Δ1-3-SS (for example, DTC319) (see FIG. 10, bar for DelA). Ceruloplasmin activity was detected using an enzymatic reaction-based colorimetric activity assay as well-known in the art (See Schosinsky et. al., Clin Chem. 1974; 20(12):1556-63). FIG. 10 shows ceruloplasmin activity in C3He-Atp7b^{tx-j} mice after receiving an intravenous injection of a vehicle control (dilution buffer, bar for WD) or an AAV infusion of ATP7B Δ1-3-SS encoded in an AAV8 vector (bar for DelA), as measured by an enzymatic reaction-based colorimetric activity assay. Ceruloplasmin activity in uninjected wild-type (WT) mice, as measured by the same enzymatic reaction-based colorimetric activity assay is also represented in the bar graph. The plot shows activity of ceruloplasmin as measured in optical density, OD as read at 540 nm. Values expressed as mean±SEM.

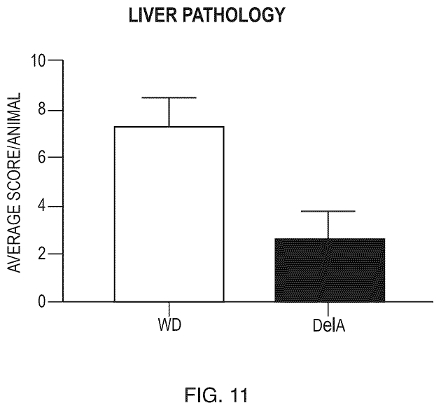

Liver was harvested from all animals in each group and stained with H&E (haematoxylin and eosin stain). H&E slides were assessed by a board certified pathologist for nuclear enlargement and hepatocellular hypertrophy, disorganization, inflammatory infiltrate, and hepatocellular necrosis according to the 0-4 scoring system. Scores from each mice in a group were averaged. FIG. 11 shows the average score as obtained after standard assessment of H&E slides for the animals in each group.

### Example 6—AAV9 Gene Therapy as a Viable Therapy for Wilson Disease (WD)

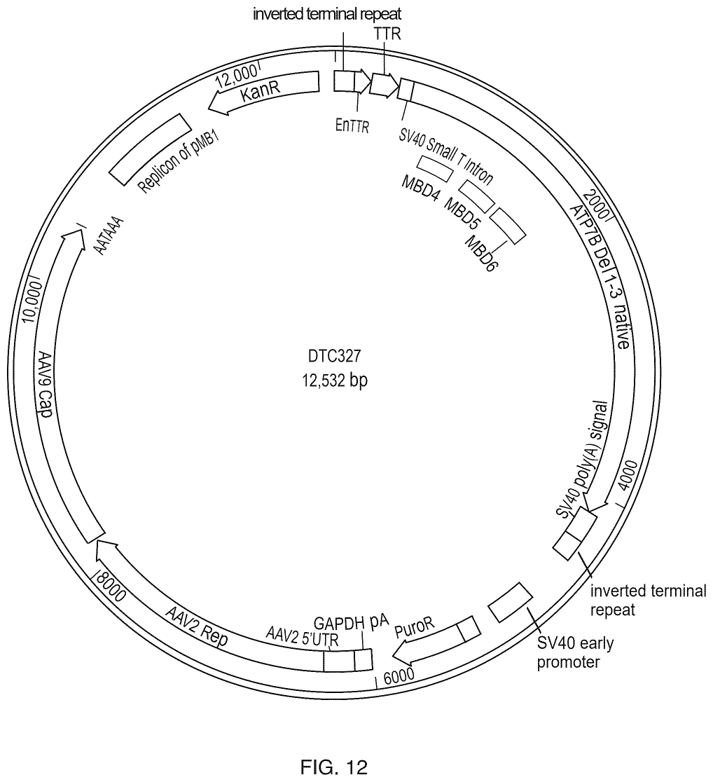

This example describes use of rAAV particles comprising ATP7B Δ1-3-SS in treating WD in a subject. An AAV vector containing a nucleotide sequence encoding a truncated human ATP7B (ATP7B Δ1-3-SS), e.g., DTC319 (FIG. 2), a Rep/Cap plasmid, which encodes four wild-type AAV2 viral replication (Rep) proteins and the three wild-type AAV VP capsid (cap) proteins from serotype 9 (AAV9), and a helper plasmid, as described in Example 1, are co-transfected into a host cell. Harvested rAAV particles are then intravenously administered to a subject in need of WD therapy. Alternatively, a subject is administered rAAV particles that are harvested from host cell transfected with vector represented by FIG. 12, for treating WD.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

1-77. (canceled)

78. A host cell comprising a recombinant nucleic acid, wherein the recombinant nucleic acid comprises a nucleic acid sequence encoding a truncated human copper-transporting ATPase 2 (ATP7B) of SEQ ID NO: 8, in which metal-binding domains (MBDs) 1-3 have been deleted, but the serine-rich loop including two serine residues (S340 and S341) between MBD3 and MBD4 is present.

79. The host cell of claim 78, wherein the recombinant nucleic acid further comprises a promoter sequence.

80. The host cell of claim 79, wherein the promoter sequence is selected from a transthyretin (TTR) promoter sequence, a chicken R-actin (CBA) promoter sequence, a cytomegalovirus (CMV) immediate early gene promoter sequence, a thyroxine binding globulin (TBG) promoter sequence, an alpha-1 anti-trypsin (A1AT) promoter sequence, and a CAG promoter sequence.

81. The host cell of claim 78, wherein the recombinant nucleic acid further comprises a 5′-inverted terminal repeat (ITR) sequence and a 3′-ITR sequence.

82. The host cell of claim 78, wherein the host cell is suitable for propagation of recombinant adeno-associated virus (rAAV) particles.

83. The host cell of claim 82, wherein the recombinant nucleic acid is stably integrated into genome of the host cell.

84. The host cell of claim 82, wherein the host cell is a HeLa cell, a HeLa S3 cell, a Cos-7 cell, a HEK293 cell, an A549 cell, a BHK cell, a Vero cell, an RD cell, an HT-1080 cell, an ARPE-19 cell, or a MRC-5 cell.

85. A method of producing a recombinant adeno-associated virus (rAAV) for use in the treatment of Wilson disease, the method comprising:
a. delivering to a eukaryotic host cell culture:
i. an AAV vector containing a nucleotide sequence encoding a truncated human copper-transporting ATPase 2 (ATP7B) of SEQ ID NO: 8, in which metal-binding domains (MBDs) 1-3 have been deleted, but the serine-rich loop including two serine residues (5340 and S341) between MBD3 and MBD4 is present;
ii. AAV rep and cap genes; and
iii. helper functions;
b. culturing the eukaryotic host cell culture under conditions to produce rAAV; and
c. harvesting rAAV particles from the eukaryotic host cell culture.

86. The method of claim 85, wherein the eukaryotic host cell culture comprises a stable cell line.

87. The method of claim 86, wherein the stable cell line comprises the AAV vector and/or the AAV rep and cap genes stably integrated into eukaryotic host cell genome.

88. The method of claim 87, wherein the helper functions are delivered to the eukaryotic host cell culture in trans.

89. The method of claim 88, wherein the helper functions are delivered by adenovirus.

90. The method of claim 85, wherein the AAV rep genes are from AAV2.

91. The method of claim 85, wherein the AAV cap genes are from AAV8 or AAV9.

92. The method of claim 85, wherein the helper functions are delivered by a helper plasmid.

93. The method of claim 85, wherein the AAV vector further comprises:
a. an AAV 5′-ITR sequence of SEQ ID NO: 2;
b. an enhancer sequence of SEQ ID NO: 3;
c. a promoter sequence of SEQ ID NO: 12;
d. a nucleic acid sequence of SEQ ID NO: 1 or SEQ ID NO: 15 encoding the truncated human ATP7B; and
e. an AAV 3′-ITR of SEQ ID NO: 2.

94. The method of claim 85, wherein the eukaryotic host cell culture comprises HeLa cells, HeLa S3 cells, Cos-7 cells, HEK293 cells, A549 cells, BHK cells, Vero cells, RD cells, HT-1080 cells, ARPE-19 cells, or MRC-5 cells.

95. A recombinant nucleic acid comprising a nucleic acid sequence which is at least 80% identical to SEQ ID NO: 14, wherein the recombinant nucleic acid comprises a nucleic acid sequence encoding a truncated human copper-transporting ATPase 2 (ATP7B) of SEQ ID NO: 8, in which metal-binding domains (MBDs) 1-3 have been deleted, but the serine-rich loop including two serine residues (S340 and S341) between MBD3 and MBD4 is present.

96. The recombinant nucleic acid of claim 95, wherein the nucleic acid sequence comprises SEQ ID NO: 14.

97. The recombinant nucleic acid of claim 95, wherein the nucleic acid sequence consists of SEQ ID NO: 14.
